(19) United States
(12) Patent Application Publication
(10) Pub. No.: US 2009/0317422 A1
(43) Pub. Date: Dec. 24, 2009

(54) USE OF PPD FOR THE ADJUVANTATION OF A NUCLEIC ACID VACCINE

(75) Inventors: Jean-Yves Bonnefoy, Strasbourg (FR); Jean-Marc Limacher, Matzenheim (FR); Stéphane Paul, Strasbourg (FR)

Correspondence Address: BUCHANAN, INGERSOLL & ROONEY PC, POST OFFICE BOX 1404, ALEXANDRIA, VA, 22313-1404, US

(73) Assignee: TRANSGENE S.A., STRASBOURG CEDEX (FR)

(21) Appl. No.: 12/304,386

(22) PCT Filed: Jun. 14, 2007
(86) PCT No.: PCT/EP2007/005261

§ 371 Date: Jun. 9, 2009

Related U.S. Application Data
(60) Provisional application No. 60/853,019, filed on Oct. 20, 2006.

(30) Foreign Application Priority Data
Jun. 20, 2006 (EP) 06360026.6

(51) Int. Cl. A61K 39/385; A61P 31/00

(52) U.S. Cl. 424/196.11; 424/197.11; 424/193.1

(57) ABSTRACT

The present invention provides a novel adjuvant for nucleic acid vaccines, and in particular the present invention provides nucleic acid vaccines that comprise, or are administered in association with PPD. The present invention also provides methods to improve the therapeutic efficacy of nucleic acid vaccines.

The present invention provides a novel adjuvant for nucleic acid vaccines, and in particular the present invention provides nucleic acid vaccines that comprise, or are administered in association with PPD. The present invention also provides methods to improve the therapeutic efficacy of nucleic acid vaccines.

More particularly, the present invention provides the use of PPD in the manufacture of a nucleic acid vaccine composition for the purpose of enhancing the immune response against the specific antigen that is encoded by the nucleic acid vaccine. Vaccine compositions, kits comprising separate nucleic acid composition and compositions comprising PPD for separate administration, methods of manufacture of the vaccines and kits, and methods of treatment of individuals with the vaccine compositions of the present invention are provided.

For years, vaccination techniques have essentially consisted in the introduction into an animal of an antigen (e.g. a protein, a killed or attenuated virus) in order to raise an immune response directed against an infectious organism. Since the end of the 80's new vaccination techniques have appeared which consist in the introduction into an animal of a vector comprising a nucleic acid sequence coding for the antigen. For example, a live vaccinia virus encoding a rabies glycoprotein has been successfully used for the Elimination of terrestrial rabies in Western European countries (Cliquet et al., Dev Biol (Basel)., 2004, 119, 185-204). The major advantage of nucleic acid immunization is that both cellular (including CD4+ and CD8+ T cells) and humoral immune responses can be induced because the encoded antigen is processed through both endogenous and exogenous pathways, and peptide epitopes are presented by major histocompatibility complexes (MHC) class I as well as class II complexes (Haupt et al., Exp Biol Med (Maywood), 2002, 227, 227-37).

The efficient generation of a CTL response has paved the way for the prophylactic or therapeutic treatment of cancer by nucleic acid vaccination. Many tumor cells express specific antigen(s) called TAA (for tumor associated antigen), but these antigens are poorly recognized by the immune system which is down regulated by factors at the periphery of tumor. The vaccination of patients with a nucleic acid encoding a TAA leads to the expression of the TAA in an environment where the immune system is fully effective and generates an immune response specifically directed against the tumor cells.

As for every treatment, there is always a need to improve the efficacy of nucleic acid vaccination. Accordingly, ways to quantitatively raise the immune response or to shift the type of response to that which would be most efficacious for the disease indication may be useful.

However the identification of adjuvants suitable for nucleic acid vaccination is not straightforward since the mechanisms implicated in the immune response against a traditional vaccine or a nucleic acid vaccine are not the same. For example, the applicant has found that strong adjuvants of traditional vaccine such as BCG, montanide, levimazole or chloroquine are unable to increase an immune response against an antigen encoded by a nucleic acid.

PPD (purified protein derivative) is a mix of compounds extracted from Mycobacterium tuberculosis. PPD is used as a test for the detection of tuberculin reactivity. After intradermal injection of PPD, the production of a delayed hypersensitivity reaction characterized by a raised bump is a sign of tuberculosis infection.

The use of PPD as a carrier for classical antigen has already been disclosed in the prior art. For example, Perraut et al. (Clin. Exp. Immunol., 1993, 93, 382-6) describes a vaccine comprising synthetic malaria peptides conjugated to PPD. Ohno et al. (US20060008478) discloses complexes comprising PPD and an antigen wherein these two components are co-precipitate. The entire prior art discloses the simultaneous injection of the PPD and of the antigen.

The applicant has found that PPD is a very potent adjuvant of nucleic acid vaccine and more particularly to nucleic acid vaccine using a recombinant virus as a vector. This discovery was particularly surprising since the various viral antigens present at the surface of the virus or expressed during the viral infection was supposed to be sufficient to adjuvant the immune response raised against the antigen (J Immunol., 2005, 175, 599-606).

### SUMMARY OF THE INVENTION

According to one embodiment of the present invention there is provided a vaccine composition comprising (i) PPD (ii) a nucleic acid sequence encoding an antigen.

In a further aspect, the invention provides a kit of part comprising (i) PPD, and (ii) a nucleic acid sequence encoding an antigen.

In a further aspect, the invention provides a method of increasing an immune response to an antigen, said method comprising administration, either sequentially or simultaneously, a nucleic acid encoding an antigen and PPD.

In a further embodiment there is provide the use of PPD in the manufacture of a medicament for the enhancement of an immune response to an antigen encoded by a nucleic acid sequence, said nucleic acid sequence being administered either sequentially or simultaneously with said derivative.

In a further embodiment the present invention further provides a pharmaceutical composition comprising PPD derivative to enhance an immune response to an antigen encoded by a nucleic acid sequence.

In another embodiment, the present invention provides a method of raising an immune response in a mammal against a disease state, comprising administering to said mammal a nucleic acid sequence encoding an antigenic peptide associated with the disease state; additionally administering PPD to said mammal to raise said immune response.

Further provided is a method of increasing the immune response of a mammal to an immunogen, comprising the step of administering to said mammal, a nucleic acid sequence encoding said immunogen, additionally administering PPD to said mammal in an amount effective to increase said immune response.

### DETAILED DESCRIPTION OF THE INVENTION

As used throughout the entire application, the terms “a” and “an” are used in the sense that they mean “at least one”, “at least a first”, “one or more” or “a plurality” of the referenced components or steps, unless the context clearly dictates otherwise. For example, the term “a cell” includes a plurality of cells, including mixtures thereof.

The term “and/or” wherever used herein includes the meaning of “and”, “or” and “all or any other combination of the elements connected by said term”.

The term “about” or “approximately” as used herein means within 20%, preferably within 10%, and more preferably within 5% of a given value or range.

As used herein, the term “comprising” is intended to mean that the products, compositions and methods include the referenced components or steps, but not excluding others. “Consisting essentially of” when used to define products, compositions and methods, shall mean excluding other components or steps of any essential significance. Thus, a composition consisting essentially of the recited components would not exclude trace contaminants and pharmaceutically acceptable carriers. “Consisting of” shall mean excluding more than trace elements of other components or steps.

As used herein the term vaccine composition refers to a combination of a nucleic acid sequence encoding an antigen, and PPD. The combination is, for example, in the form of an admixture of the two components in a single pharmaceutically acceptable formulation or in the form of separate, individual components, for example in the form of a kit comprising a nucleic acid sequence encoding an antigen, and PPD, wherein the two components are for separate, sequential or simultaneous administration. Preferably, the administration of the two components is substantially simultaneous.

As used herein, the term “PPD”, “Purified Protein Derivative” or “tuberculin” (which can be used interchangeably) refers to the proteins obtained by the Seibert Process (Seibert et al. Am. Rev. Tuberc., 1934, 30, 713-720 and Seibert et al. Am. Rev. Tuberc., 1941, 44, 9-23). PPD also refers to compositions comprising the protein obtained by the Seibert process. Such compositions are, for example, commercially available under the Applisol® (Parkedale Pharmaceuticals, Rochester, USA), PPD Tine Test® (Lederlele Pharmaceutical, Pearl River, USA), Tubertest (Sanofi Pasteur Msd), Tubersol® (Aventis Pasteur), Aplitest®, Sclavo Test-PPD® (Sclavo Laboratories, Italy), or Mono-Vacc Test (O.T.) brands. According to a preferred embodiment of the invention, PPD is a composition chosen from the group comprising tubertest and tubersol.

It is possible for the vaccination methods and compositions according to the present application to be adapted for protection or treatment of mammals against a variety of disease states such as, for example, viral, bacterial or parasitic infections, cancer, allergies and autoimmune disorders.

The term “antigen” refers to a molecule or a portion of a molecule capable of being bound by a selective binding agent, such as an antibody, and additionally capable of being used in an animal to produce antibodies capable of binding to an epitope of that antigen. An antigen may have one or more epitopes.

In one embodiment, the antigen is a tumour associated antigen (TAA). TAA refers to a molecule that is detected at a higher frequency or density in tumor cells than in non-tumor cells of the same tissue type. Examples of TAA includes but are not limited to CEA, MART-1, MAGE-1, MAGE-3, GP-100, MUC-1, MUC-2, pointed mutated ras oncogene, normal or point mutated p53, overexpressed p53, CA-125, PSA, C-erb/B2, BRCA I, BRCA II, PSMA, tyrosinase, TRP-1, TRP-2, NY-ESO-1, TAG72, KSA, HER-2/neu, bcr-abl, pax3-fkhr, ews-fli-1, survivin and LRP. According to a preferred embodiment the TAA is MUC1.

In another embodiment of the invention, the antigen is a microbial antigen. A microbial antigen as used herein is an antigen of a microorganism including but not limited to virus, bacteria, parasites, and fungi.

Virus comprises but are not limited to Retroviridae, Picornaviridae (e.g. polio viruses, hepatitis A virus; enteroviruses, human Coxsackie viruses, rhinoviruses, echoviruses); Calciviridae (e.g. strains that cause gastroenteritis); Togaviridae (e.g. equine encephalitis viruses, rubella viruses); Flaviridae (e.g. dengue viruses, encephalitis viruses, yellow fever viruses); Coronoviridae (e.g. coronaviruses); Rhabdoviradae (e.g. vesicular stomatitis viruses, rabies viruses); Filoviridae (e.g. ebola viruses); Paramyxoviridae (e.g. parainfluenza viruses, mumps virus, measles virus, respiratory syncytial virus); Orthomyxoviridae (e.g. influenza viruses); Bungaviridae (e.g. Hantaan viruses, bunga viruses, phleboviruses and Nairo viruses); Arena viridae (hemorrhagic fever viruses); Reoviridae (e.g. reoviruses, orbiviurses and rotaviruses); Birnaviridae; Hepadnaviridae (Hepatitis B virus); Parvovirida (parvoviruses); Papovaviridae (papilloma viruses, polyoma viruses); Adenoviridae (most adenoviruses); Herpesviridae (herpes simplex virus (HSV) 1 and 2, varicella zoster virus, cytomegalovirus (CMV), herpes virus; Poxyiridae (variola viruses, vaccinia viruses, pox viruses); and Iridoviridae (e.g. African swine fever virus).

According to preferred embodiment of the invention said antigen is an antigen of the Human Papilloma Virus (HPV), According to a preferred embodiment, said HPV antigen is derived from HPV-16 or/and HPV-18.

According to an even more preferred embodiment, said HPV antigen is selected in the group consisting of E6 early coding region of HPV, E7 early coding region of HPV and part or combination thereof.

The present invention encompasses the use of any HPV E6 polypeptide which binding to p53 is altered or at least significantly reduced and/or the use of any HPV E7 polypeptide which binding to Rb is altered or at least significantly reduced (Munger et al., 1989, EMBO J. 8, 4099-4105; Crook et al., 1991, Cell 67, 547-556; Heck et al., 1992, Proc. Natl. Acad. Sci. USA 89, 4442-4446; Phelps et al., 1992, J. Virol. 66, 2148-2427). A non-oncogenic HPV-16 E6 variant which is suitable for the purpose of the present invention is deleted of one or more amino acid residues located from approximately position 118 to approximately position 122 (+1 representing the first methionine residue of the native HPV-16 E6 polypeptide), with a special preference for the complete deletion of residues 118 to 122 (CPEEK). A non-oncogenic HPV-16 E7 variant which is suitable for the purpose of the present invention is deleted of one or more amino acid residues located from approximately position 21 to approximately position 26 (+1 representing the first amino acid of the native HPV-16 E7 polypeptide, with a special preference for the complete deletion of residues 21 to 26 (DLYCYE). According to a preferred embodiment, the one or more HPV-16 early polypeptide(s) in use in the invention is/are further modified so as to improve MHC class I and/or MHC class II presentation, and/or to stimulate anti-HPV immunity. HPV E6 and E7 polypeptides are nuclear proteins and it has been previously shown that membrane presentation permits to improve their therapeutic efficacy (see for example WO99/03885). Thus, it may be advisable to modify at least one of the HPV early polypeptide(s) so as to be anchored to the cell membrane. Membrane anchorage can be easily achieved by incorporating in the HPV early polypeptide a membrane-anchoring sequence and if the native polypeptide lacks it a secretory sequence (i.e. a signal peptide). Membrane-anchoring and secretory sequences are known in the art. Briefly, secretory sequences are present at the N-terminus of the membrane presented or secreted polypeptides and initiate their passage into the endoplasmic reticulum (ER). They usually comprise 15 to 35 essentially hydrophobic amino acids which are then removed by a specific ER-located endopeptidase to give the mature polypeptide. Membrane-anchoring sequences are usually highly hydrophobic in nature and serves to anchor the polypeptides in the cell membrane (see for example Branden and Tooze, 1991, in Introduction to Protein Structure p. 202-214, NY Garland).

The choice of the membrane-anchoring and secretory sequences which can be used in the context of the present invention is vast. They may be obtained from any membrane-anchored and/or secreted polypeptide comprising it (e.g. cellular or viral polypeptides) such as the rabies glycoprotein, of the HIV virus envelope glycoprotein or of the measles virus F protein or may be synthetic. The membrane anchoring and/or secretory sequences inserted in each of the early HPV-16 polypeptides used according to the invention may have a common or different origin. The preferred site of insertion of the secretory sequence is the N-terminus downstream of the codon for initiation of translation and that of the membrane-anchoring sequence is the C-terminus, for example immediately upstream of the stop codon.

The HPV E6 polypeptide in use in the present invention is preferably modified by insertion of the secretory and membrane-anchoring signals of the measles F protein. Optionally or in combination, the HPV E7 polypeptide in use in the present invention is preferably modified by insertion of the secretory and membrane-anchoring signals of the rabies glycoprotein.

Bacteria comprise gram positive and gram negative bacteria. Such gram positive bacteria include, but are not limited to, Pasteurella species, Staphylococci species, and Streptococcus species. Gram negative bacteria include, but are not limited to, Escherichia coli, Pseudomonas species, and Salmonella species. Specific examples of infectious bacteria include but are not limited to, Helicobacter pyloris, Borelia burgdorferi, Legionella pneumophilia, Mycobacteria sps (e.g. M. tuberculosis, M. avium, M. intracellulare, M. kansaii, M. gordonae), Staphylococcus aureus, Neisseria gonorrhoeae, Neisseria meningitidis, Listeria monocytogenes, Streptococcus pyogenes (Group A Streptococcus), Streptococcus agalactiae (Group B Streptococcus), Streptococcus (viridans group), Streptococcus faecalis, Streptococcus bovis, Streptococcus (anaerobic sps.), Streptococcus pneumoniae, pathogenic Campylobacter sp., Enterococcus sp., Haemophilus influenzae, Bacillus antracis, corynebacterium diphtheriae, corynebacterium sp., Erysipelothrix rhusiopathiae, Clostridium perfringens, Clostridium tetani, Enterobacter aerogenes, Klebsiella pneumoniae, Pasturella multocida, Bacteroides sp., Fusobacterium nucleatum, Streptobacillus moniliformis, Treponema pallidium, Treponema pertenue, Leptospira, Rickettsia, and Actinomyces israelli.

Fungi notably comprise Cryptococcus neoformans, Histoplasma capsulatum, Coccidioides immitis, Blastomyces dermatitidis, Chlamydia trachomatis, Candida albicans.

According to another embodiment of the invention, the antigen is an antigen of an infectious organisms comprising Plasmodium such as Plasmodium falciparum, Plasmodium malariae, Plasmodium ovale, and Plasmodium vivax and Toxoplasma gondii. Blood-borne and/or tissues parasites include Plasmodium spp., Babesia microti, Babesia divergens, Leishmania tropica, Leishmania spp., Leishmania braziliensis, Leishmania donovani, Trypanosoma gambiense and Trypanosoma rhodesiense (African sleeping sickness), Trypanosoma cruzi (Chagas' disease), and Toxoplasma gondii.

According to another embodiment, the antigen is an allergen. An allergen refers to a substance that can induce an allergic or asthmatic response in a susceptible subject. The list of allergens is enormous and can include pollens, insect venoms, animal dander dust, fungal spores and drugs (e.g. penicillin). Examples of natural, animal and plant allergens include but are not limited to proteins specific to the following genuses: Canine (Canis familiaris); Dermatophagoides (e.g. Dermatophagoides farinae); Felis (Felis domesticus); Ambrosia (Ambrosia artemiisfolia; Lolium (e.g. Lolium perenne or Lolium multiflorum); Cryptomeria (Cryptomeria japonica); Alternaria (Alternaria alternata); Alder; Alnus (Alnus gultinoasa); Betula (Betula verrucosa); Quercus (Quercus alba); Olea (Olea europa); Artemisia (Artemisia vulgaris); Plantago (e.g. Plantago lanceolata); Parietaria (e.g. Parietaria officinalis or Parietaria judaica); Blattella (e.g. Blattella germanica); Apis (e.g. Apis multiflorum); Cupressus (e.g. Cupressus sempervirens, Cupressus arizonica and Cupressus macrocarpa); Juniperus (e.g. Juniperus sabinoides, Juniperus virginiana, Juniperus communis and Juniperus ashei); Thuya (e.g. Thuya orientalis); Chamaecyparis (e.g. Chamaecyparis obtusa); Periplaneta (e.g. Periplaneta americana); Agropyron (e.g. Agropyron repens); Secale (e.g. Secale cereale); Triticum (e.g. Triticum aestivum); Dactylis (e.g. Dactylis glomerata); Festuca (e.g. Festuca elatior); Poa (e.g. Poa pratensis or Poa compressa); Avena (e.g. Avena sativa); Holcus (e.g. Holcus lanatus); Anthoxanthum (e.g. Anthoxanthum odoratum); Arrhenatherum (e.g. Arrhenatherum elatius); Agrostis (e.g. Agrostis alba); Phleum (e.g. Phleum pratense); Phalaris (e.g. Phalaris arundinacea); Paspalum (e.g. Paspalum notatum); Sorghum (e.g. Sorghum halepensis); and Bromus (e.g. Bromus inermis).

As used herein, the term “immune response” encompasses B cell-mediated, T-cell mediated, or a combination of both B- and T-cell mediated responses.

The term “nucleic acid sequence” refers to a linear sequence of nucleotides. The nucleotides are either a linear sequence of polyribonucleotides or polydeoxyribonucleotides, or a mixture of both. Examples of polynucleotides in the context of the present invention include—single and double stranded DNA, single and double stranded RNA, and hybrid molecules that have both mixtures of single and double stranded DNA and RNA. Further, the polynucleotides of the present invention may have one or more modified nucleotides.

According to a preferred embodiment of the invention, the nucleic acid sequence encoding an antigen is comprised in a vector.

The vector can be of plasmid or viral origin and can, where appropriate, be combined with one or more substances which improve the transfectional efficiency and/or stability of the vector. These substances are widely documented in the literature which is available to the skilled person (see, for example, Feigner et al., 1987, Proc. West. Pharmacol. Soc. 32, 115-121; Hodgson and Solaiman, 1996, Nature Biotechnology 14, 339-342; Remy et al., 1994, Bioconjugate Chemistry, 5, 647-654). By way of non-limiting illustration, the substances can be polymers, lipids, in particular cationic lipids, liposomes, nuclear proteins or neutral lipids. These substances can be used alone or in combination. A combination which can be envisaged is that of a recombinant plasmid vector which is combined with cationic lipids (DOGS, DC-CHOL, spermine-chol, spermidine-chol, etc.), lysophospholipides (for example Hexadecylphosphocholine) and neutral lipids (DOPE).

According to a preferred embodiment, the cationic lipids which can be used in the present invention are the cationic lipids describes in EP901463B1 and more preferably pcTG90.

The choice of the plasmids which can be used within the context of the present invention is immense. They can be cloning vectors and/or expression vectors. In a general manner, they are known to the skilled person and, while a number of them are available commercially, it is also possible to construct them or to modify them using the techniques of genetic manipulation. Examples which may be mentioned are the plasmids which are derived from pBR322 (Gibco BRL), pUC (Gibco BRL), pBluescript (Stratagene), pREP4, pCEP4 (Invitrogene) or p Poly (Lathe et al., 1987, Gene 57, 193-201). Preferably, a plasmid which is used in the context of the present invention contains an origin of replication which ensures that replication is initiated in a producer cell and/or a host cell (for example, the ColE1 origin will be chosen for a plasmid which is intended to be produced in E. coli and the oriP/EBNA1 system will be chosen if it desired that the plasmid should be self-replicating in a mammalian host cell, Lupton and Levine, 1985, Mol. Cell. Biol. 5, 2533-2542; Yates et al., Nature 313, 812-815). The plasmid can additionally comprise a selection gene which enables the transfected cells to be selected or identified (complementation of an auxotrophic mutation, gene encoding resistance to an antibiotic, etc.). Naturally, the plasmid can contain additional elements which improve its maintenance and/or its stability in a given cell (cer sequence, which promotes maintenance of a plasmid in monomeric form (Summers and Sherrat, 1984, Cell 36, 1097-1103, sequences for integration into the cell genome).

With regard to a viral vector, it is possible to envisage a vector which is derived from a poxvirus (vaccinia virus, in particular MVA, canarypoxvirus, etc.), from an adenovirus, from a retrovirus, from a herpesvirus, from an alphavirus, from a foamy virus or from an adenovirus-associated virus. It is possible to use replication competent or replication deficient viral vectors. Preference will be given to using a vector which does not integrate. In this respect, adenoviral vectors and vectors deriving from poxvirus and more preferably vaccinia virus and MVA are very particularly suitable for implementing the present invention.

According to a preferred embodiment, the viral vector according to the invention derives from a Modified Vaccinia Virus Ankara (MVA). MVA vectors and methods to produce such vectors are fully described in European patents EP83286 and EP206920, as well as in Mayr et al. (1975, Infection 3, 6-14) and Sutter et Moss (1992, Proc. Natl. Acad. Sci. USA 89, 10847-10851). According to a more preferred embodiment, the nucleic acid sequence according to the invention may be inserted in deletion I, II, III, IV, V and VI of the MVA vector and even more preferably in deletion III (Meyer et al., 1991, J. Gen. Virol. 72, 1031-1038; Sutter et al., 1994, Vaccine 12, 1032-1040).

Retroviruses have the property of infecting, and in most cases integrating into, dividing cells and in this regard are particularly appropriate for use in relation to cancer. A recombinant retrovirus according to the invention generally contains the LTR sequences, an encapsidation region and the nucleotide sequence according to the invention, which is placed under the control of the retroviral LTR or of an internal promoter such as those described below. The recombinant retrovirus can be derived from a retrovirus of any origin (murine, primate, feline, human, etc.) and in particular from the M0MuLV (Moloney murine leukemia virus), MVS (Murine sarcoma virus) or Friend murine retrovirus (Fb29). It is propagated in an encapsidation cell line which is able to supply in trans the viral polypeptides gag, pol and/or env which are required for constituting a viral particle. Such cell lines are described in the literature (PA317, Psi CRIP GP+Am-12 etc.). The retroviral vector according to the invention can contain modifications, in particular in the LTRs (replacement of the promoter region with a eukaryotic promoter) or the encapsidation region (replacement with a heterologous encapsidation region, for example the VL3O type) (see French applications 94 08300 and 97 05203)

Preference will be also given to using an adenoviral vector which lacks all or part of at least one region which is essential for replication and which is selected from the E1, E2, E4 and L1-L5 regions in order to avoid the vector being propagated within the host organism or the environment. A deletion of the E1 region is preferred. However, it can be combined with (an)other modification(s)-/deletion(s) affecting, in particular, all or part of the E2, E4 and/or L1-L5 regions, to the extent that the defective essential functions are complemented in trans by means of a complementing cell line and/or a helper virus. In this respect, it is possible to use second-generation vectors of the state of the art (see, for example, international applications WO-A-94/28152 and WO-A-97/04119). By way of illustration, deletion of the major part of the E1 region and of the E4 transcription unit is very particularly advantageous. For the purpose of increasing the cloning capacities, the adenoviral vector can additionally lack all or part of. the nonessential E3 region. According to another alternative, it is possible to make use of a minimal adenoviral vector which retains the sequences which are essential for encapsidation, namely the 5′ and 3′ ITRs (Inverted Terminal Repeat), and the encapsidation region. The various adenoviral vectors, and the techniques for preparing them, are known (see, for example, Graham and Prevect, 1991, in Methods in Molecular Biology, Vol 7, p 109-128; Ed: E. J. Murey, The Human Press mc).

Furthermore, the origin of the adenoviral vector according to the invention can vary both from the point of view of the species and from the point of view of the serotype. The vector can be derived from the genome of an adenovirus of human or animal (canine, avian, bovine, murine, ovine, porcine, simian, etc.) origin or from a hybrid which comprises adenoviral genome fragments of at least two different origins. More particular mention may be made of the CAV-I or CAV-2 adenoviruses of canine origin, of the DAV adenovirus of avian origin or of the Bad type 3 adenovirus of bovine origin (Zakharchuk et al., Arch. Virol., 1993, 128: 171-176; Spibey and Cavanagh, J. Gen. Virol. 1989, 70: 165-172; Jouvenne et al., Gene, 1987, 60: 21-28; Mittal et al., J. Gen. Virol., 1995, 76: 93-102). However, preference will be given to an adenoviral vector of human origin which is preferably derived from a serotype C-adenovirus, in particular a type 2 or 5 serotype C adenovirus.

The term “replication-competent” as used herein refers to a viral vector capable of replicating in a host cell in the absence of any trans-complementation.

According to a preferred embodiment of the invention, the replication competent vector is a replication competent adenoviral vector. These replication competent adenoviral vectors are well known by the one skilled in the art. Among these, adenoviral vectors deleted in the E1b region coding the 55 kD P53 inhibitor, as in the ONYX-015 virus (Bischoff et al, 1996; Heise et al., 2000; WO 94/18992), are particularly preferred. Accordingly, this virus can be used to selectively infect and kill p53-deficient neoplastic cells. A person of ordinary skill in the art can also mutate and disrupt the p53 inhibitor gene in adenovirus 5 or other viruses according to established techniques. Adenoviral vectors deleted in the E1A Rb binding region can also be used in the present invention. For example, Delta24 virus which is a mutant adenovirus carrying a 24 base pair deletion in the E1A region (Fueyo et al., 2000). Delta24 has a deletion in the Rb binding region and does not bind to Rb. Therefore, replication of the mutant virus is inhibited by Rb in a normal cell. However, if Rb is inactivated and the cell becomes neoplastic, Delta24 is no longer inhibited. Instead, the mutant virus replicates efficiently and lyses the Rb-deficient cell.

An adenoviral vector according to the present invention can be generated in vitro in Escherichia coli (E. coli) by ligation or homologous recombination (see, for example, international application WO-A-96/17070) or else by recombination in a complementing cell line.

According to a preferred embodiment of the invention, the vector further comprises the elements necessary for the expression of the antigen.

The elements necessary for the expression consist of all the elements which enable the nucleic acid sequence to be transcribed into RNA and the mRNA to be translated into polypeptide. These elements comprise, in particular, a promoter which may be regulable or constitutive. Naturally, the promoter is suited to the chosen vector and the host cell. Examples which may be mentioned are the eukaryotic promoters of the PGK (phosphoglycerate kinase), MT (metallothionein; Mclvor et al., 1987, Mol. Cell Biol. 7, 838-848), α-1 antitrypsin, CFTR, surfactant, immunoglobulin, actin (Tabin et al., 1982, Mol. Cell Biol. 2, 426-436) and SRa (Takebe et al., 1988, Mol. Cell Biol. 8, 466-472) genes, the early promoter of the SV40 virus (Simian virus), the LTR of RSV (Rous sarcoma virus), the HSV-I TK promoter, the early promoter of the CMV virus (Cytomegalovirus)., the p7.5K pH5R, pK1L, p28 and p11 promoters of the vaccinia virus, and the E1A and MLP adenoviral promoters. The promoter can also be a promoter which stimulates expression in a tumor or cancer cell. Particular mention may be made of the promoters of the MUC-I gene, which is overexpressed in breast and prostate cancers (Chen et al., 1995, J. Clin. Invest. 96, 2775-2782), of the CEA (standing for carcinoma embryonic antigen) gene, which is overexpressed in colon cancers (Schrewe et al., 1990, Mol. Cell. Biol. 10, 2738-2748) of the tyrosinase gene, which is overexpressed in melanomas (Vile et al., 1993, Cancer Res. 53, 3860-3864), of the ERBB-2 gene, which is overexpressed in breast and pancreatic cancers (Harris et al., 1994, Gene Therapy 1, 170-175) and of the α-fetoprotein gene, which is overexpressed in liver cancers (Kanai et al., 1997, Cancer Res. 57, 461-465). The cytomegalovirus (CMV) early promoter is very particularly preferred.

However, when a vector deriving from a Vaccinia Virus (as for example an MVA vector) is used, the promoter of the thymidine kinase 7.5K gene is particularly preferred.

The necessary elements can furthermore include additional elements which improve the expression of the nucleotide sequence according to the invention or its maintenance in the host cell. Intron sequences, secretion signal sequences, nuclear localization sequences, internal sites for the reinitiation of translation of IRES type, transcription termination poly A sequences, tripartite leaders and origins of replication may in particular be mentioned. These elements are known to the skilled person.

The recombinant vector according to the invention can also comprise one or more additional genes of interest, with it being possible for these genes to be placed under the control of the same regulatory elements (polycistronic cassette) or of independent elements. Genes which may in particular be mentioned are the genes encoding interleukins IL-2, IL-4, IL-7, IL-10, IL-12, IL-15, IL-18, chemokines as CCL19, CCL20, CCL21, CXCL-14, interferons, tumor necrosis factor (TNF), colony stimulating factors (CSF), in particular GM-CSF, and factors acting on innate immunity and angiogenesis (for example PAI-1, standing for plasminogen activator inhibitor). In one particular embodiment, the recombinant vector according to the invention comprises the gene of interest encoding IL-2.

The present invention further provides a pharmaceutical composition comprising a vaccine composition, a kit of parts according to the present invention, and a pharmaceutically acceptable carrier.

The present invention further provides a process for the manufacture of a vaccine composition comprising mixing PPD with a nucleic acid encoding an antigen.

In a further embodiment, the process further provides incorporating the vaccine composition within a pharmaceutically acceptable carrier.

The pharmaceutically acceptable carrier is preferably isotonic, hypotonic or weakly hypertonic and has a relatively low ionic strength, such as for example a sucrose solution. Moreover, such a carrier may contain any solvent, or aqueous or partially aqueous liquid such as nonpyrogenic sterile water. The pH of the pharmaceutical composition is, in addition, adjusted and buffered so as to meet the requirements of use in vivo. The pharmaceutical composition may also include a pharmaceutically acceptable diluent, adjuvant or excipient, as well as solubilizing, stabilizing and preserving agents. For injectable administration, a formulation in aqueous, nonaqueous or isotonic solution is preferred. It may be provided in a single dose or in a multidose in liquid or dry (powder, lyophilisate and the like) form which can be reconstituted at the time of use with an appropriate diluent.

When the nucleic acid sequence encoding an antigen is of plasmid origin, the pharmaceutically acceptable carrier is preferably a particle usable for gene gun administration. For example, said carrier may be a gold bead.

The present invention further provides a method of treating a patient suffering from or susceptible to a tumor, by the administration of a vaccine composition, a kit of part or pharmaceutical composition according to the invention. The tumor to be treated may be carcinoma of the breast; carcinoma of the lung, including non-small cell lung carcinoma; or prostate, gastric, and other gastrointestinal carcinomas.

The present invention further provides a method of treating a patient suffering from or susceptible to an infectious disease, by the administration of a vaccine composition, a kit of part or pharmaceutical composition as herein described. The term “infectious disease” as used herein, includes, but is not limited to any disease that is caused by an infectious organism. Infectious organisms may comprise viruses, (e.g., single stranded RNA viruses, single stranded DNA viruses, human immunodeficiency virus (HIV), hepatitis A, B, and C virus, herpes simplex virus (HSV), cytomegalovirus (CMV) Epstein-Barr virus (EBV), human papilloma virus (HPV)), parasites (e.g., protozoan and metazoan pathogens such as Plasmodia species, Leishmania species, Schistosoma species, Trypanosoma species), bacteria (e.g., Mycobacteria, in particular, M. tuberculosis, Salmonella, Streptococci, E. coli, Staphylococci), fungi (e.g., Candida species, Aspergillus species), Pneumocystis carinii, and prions.

The present invention further provides a method of treating a patient suffering from or susceptible to allergy, by the administration a vaccine composition, a kit of part or pharmaceutical composition as herein described.

The present invention further provides a method for increasing an immune response in a mammal to an antigen, the method comprising the administration to the mammal of the following components: (i) PPD, (ii) a nucleic acid sequence encoding an antigen. In one embodiment, the method comprises simultaneous administration of any two components (i) and (ii). Alternatively, the method comprises sequential administration of components (i) and (ii).

As used herein, the term “sequential” means that the components are administered to the subject one after another within a timeframe. Thus, sequential administration may permit one component to be administered within 5 minutes, 10 minutes or a matter of hours after the other.

The present invention further provides a method of raising an immune response in a mammal against a disease state, comprising administering to the mammal a nucleic acid sequence encoding an antigen with the disease state and further administering to the mammal PPD to raise the immune response.

The present invention further provides a method of increasing the immune response of a mammal to an antigen, comprising the step of administering to the mammal within a nucleic acid sequence encoding the antigen and further administering PPD to the mammal.

The present invention further provides use of PPD in the manufacture of a medicament for enhancing immune responses initiated by an antigen being expressed as a result of administration to a mammal of a nucleic acid sequence encoding the antigen.

The present invention further provides the use of PPD for the manufacture of medicaments for concomitant or sequential administration to a mammal for the enhancement of an immune response to an antigen encoded by a nucleic acid sequence, in which said nucleic acid sequence is formulated into a separate medicament.

Administering the vaccine composition, the kit of part or the pharmaceutical composition of the present invention may be accomplished by any means known to the skilled artisan. Preferred routes of administration include but are not limited to intradermal, subcutaneous, oral, parenteral, intramuscular, intranasal, sublingual, intratracheal, inhalation, ocular, vaginal, and rectal. According to a preferred embodiment, the vaccine composition, the kit of part or the pharmaceutical composition of the present invention are delivered subcutaneously or intradermally. According to an even more preferred embodiment of the invention, PPD and the nucleic acid encoding an antigen are administered at the same site.

The administration may take place in a single dose or a dose repeated one or several times after a certain time interval. Desirably, the vaccine composition, the kit of part or the pharmaceutical composition are administered 1 to 10 times at weekly intervals.

The dose of administration of PPD will vary, but may be from 0.1 UI to 50 UI, advantageously from 1 UI to 10 UI and even more advantageously about 5 UI.

The dose of administration of the nucleic acid sequence encoding a antigen will also vary, and can be adapted as a function of various parameters, in particular the mode of administration; the composition employed; the age, health, and weight of the host organism; the nature and extent of symptoms; kind of concurrent treatment; the frequency of treatment; and/or the need for prevention or therapy. Further refinement of the calculations necessary to determine the appropriate dosage for treatment is routinely made by a practitioner, in the light of the relevant circumstances. For general guidance, suitable dosage for a MVA-containing composition varies from about 10⁴ to 10¹⁰ pfu (plaque forming units), desirably from about 10⁵ and 10⁸ pfu whereas adenovirus-comprising composition varies from about 10⁵ to 10¹³ iu (infectious units), desirably from about 10⁷ and 10¹² iu. A composition based on vector plasmids may be administered in doses of between 10 μg and 20 mg, advantageously between 100 μg and 2 mg. Preferably the composition is administered at dose(s) comprising from 5 10⁵ pfu to 5 10⁷ pfu of MVA vaccinia vector.

When the use or the method according to the invention is for the treatment of cancer, the method or use of the invention can be carried out in conjunction with one or more conventional therapeutic modalities (e.g. radiation, chemotherapy and/or surgery). The use of multiple therapeutic approaches provides the patient with a broader based intervention. In one embodiment, the method of the invention can be preceded or followed by a surgical intervention. In another embodiment, it can be preceded or followed by radiotherapy (e.g. gamma radiation). Those skilled in the art can readily formulate appropriate radiation therapy protocols and parameters which can be used (see for example Perez and Brady, 1992, Principles and Practice of Radiation Oncology, 2nd Ed. JB Lippincott Co; using appropriate adaptations and modifications as will be readily apparent to those skilled in the field).

The present Invention further concerns a method for improving the treatment of a cancer patient which is undergoing chemotherapeutic treatment with a chemotherapeutic agent, which comprises co-treatment of said patient along with a method as above disclosed.

The present Invention further concerns a method of improving http://www.micropat.com/perl/di/psrecord.pl?ticket=037405101546&listid=114934200603310905&container_id=763883&patnum=US6015827A cytotoxic effectiveness of cytotoxic drugs or radiotherapy which comprises co-treating a patient in need of such treatment along with a method as above disclosed.

When the use or the method according to the invention is for the treatment of an infectious disease, the method or use of the invention can be carried out with the use or another therapeutic compounds such as antibiotics, antifungal compounds and/or antiviral compounds.

The present Invention further concerns a method of improving http://www.micropat.com/perl/di/psrecord.pl?ticket=037405101546&listid=114934200603310905&container_id=763883&patnum=US6015827A the therapeutic efficacy of an antibiotic, an antiviral or an antifungal drug which comprises co-treating a patient in need of such treatment along with a method as above disclosed.

In another embodiment, the method or use of the invention is carried out according to a prime boost therapeutic modality which comprises sequential administration of one or more primer composition(s) and one or more booster composition(s). Typically, the priming and the boosting compositions use different vehicles which comprise or encode at least an antigenic domain in common. The priming composition is initially administered to the host organism and the boosting composition is subsequently administered to the same host organism after a period varying from one day to twelve months. The method of the invention may comprise one to ten sequential administrations of the priming composition followed by one to ten sequential administrations of the boosting composition. Desirably, injection intervals are a matter of one week to six months. Moreover, the priming and boosting compositions can be administered at the same site or at alternative sites by the same route or by different routes of administration.

The examples which follow are intended to illustrate the various subjects of the present invention and are consequently not limiting in character.

All of the above cited disclosures of patents, publications and database entries are specifically incorporated herein by reference in their entirety to the same extent as if each such individual patent, publication or entry were specifically and individually indicated to be incorporated by reference.

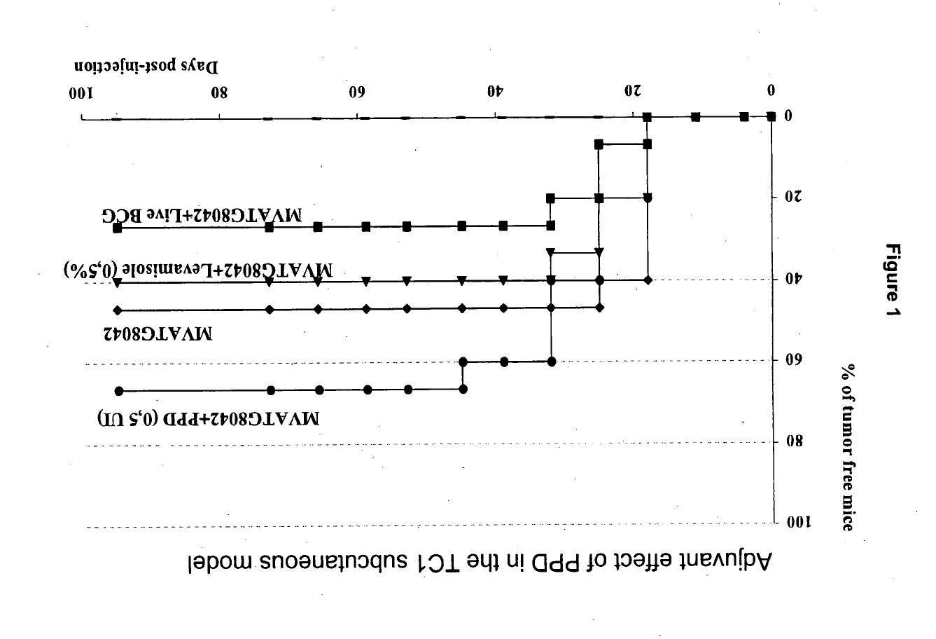

FIG. 1 depicts the percentage of tumor free mice after injection of the TC1 tumor cells expressing the E6 and E7 protein of HPV16. After injection of the TC1 cells, the mice were vaccinated three times (at weekly intervals) with a MVA vector expressing the E6/E7 antigen in conjunction with PPD, live BCG or levimasole. An empty MVA and a MVA encoding the E6/E7 antigen injected alone were used as controls.

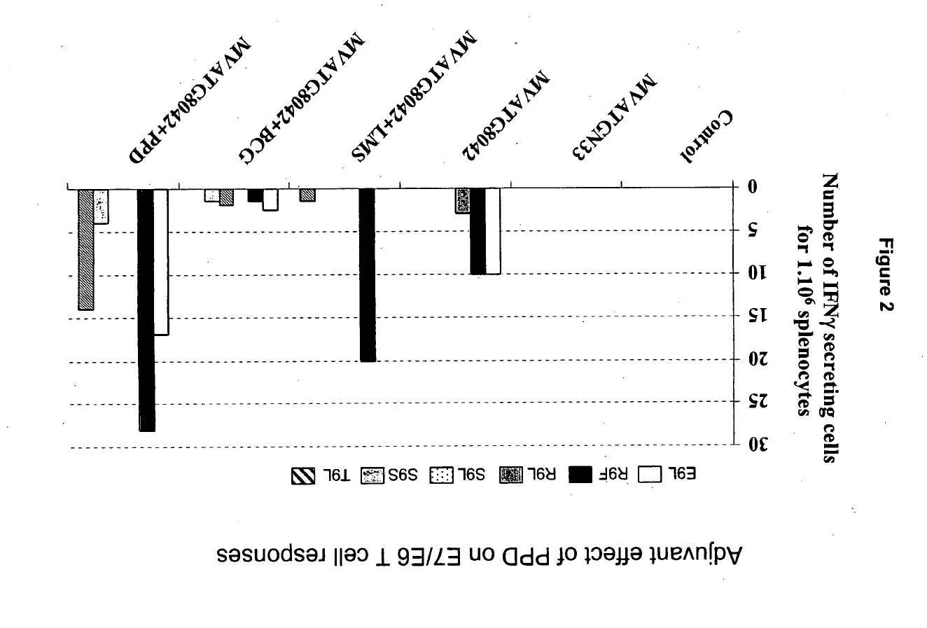

FIG. 2 depicts the number of T cell specific for E6/E7 epitopes after immunisation of mice with an MVA vector encoding the E6/E7 antigen in conjunction with the subcutaneous injection of levimasole, live BCG or PPD.

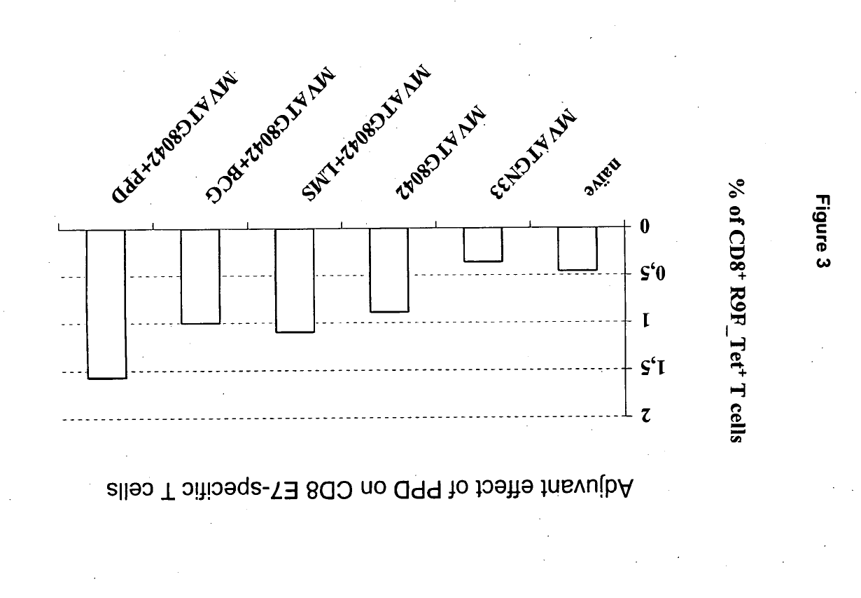

FIG. 3 depicts the percentage of CD8+ T cells, specific for an E7 peptide (R9F), after immunisation of mice with an MVA vector encoding the E6/E7 antigen in conjunction with the subcutaneous injection of levimasole, live BCG or PPD.

### EXAMPLES

### 1. Test Article

a. Denomination and Brief Description of Each Vector Construction

| | Batch | | | |
|---|---|---|---|---|
| Virus | concentration | E6tm/E7tm | hIL-2 | |
| Denomination | (pfu/ml) | prom | prom | Transgene |
| MVAN33 | 4, 5.109 pfu/ml | — | — | — |
| MVATG8042 | 3, 1.109 pfu/ml | P7.5 | PH5R | E6/E7; |
| | | | | hIL-2 |

b. Conditions of Storage:

Viruses were received from the Molecular Immunology Department and then were maintained at −80° C. until the day of injection. The viral suspension was rapidly thawed immediately prior to dilution and administration.

### 2. Animal Model

a. Species/Strain/Supplier:

SPF healthy female C57BI/6 mice were obtained from Charles River (Les Oncins, France).

Specification: The animals were 6-weeks-old upon arrival. At the beginning of experimentation, they were 7-week-old.

Environment: The animals were housed in a single, exclusive room, air-conditioned to provide a minimum of 11 air changes per hour. The temperature and relative humidity ranges were within 20° C. and 24° C. and 40 to 70% respectively. Lighting was controlled automatically to give a cycle of 12 hours of light and 12 hours of darkness.

Specific pathogen free status was checked by regular control of sentinel animals.

Diet: Throughout the study the animals had access ad libitum to sterilized diet type RM1 (Dietex France, Saint Gratien). Sterile water was provided ad libitum via bottles.

### 3. Cells Description

a. Cells Characteristics/Conditions of Use:

TC1 tumor cells: These cells obtained from C57BI6 mice lung, have been transduced with 2 retroviruses: LXSN16E6E7 expressing E6 and E7 from HPV16 and pVEJB expressing the ras gene. There are a kind gift of Dr TC Wu (The Johns Hopkins University, Baltimore, USA). The cells were cultured in DMEM containing 0.5 mg/ml G418 and 0.2 mg/ml Hygromycine. Adherents cells were removed by trypsine treatment and after 3 washings, tumor challenge were performed subcutaneously with 2.105 TC1 viable cells.

### 4. Protocol

a. Immunizations Schedule

For the immunotherapeutic experiments, 15 C57BI6 female mice were challenged subcutaneously in the right flank with 2.10⁵ TC1 cells at D1. Mice were treated three times, subcutaneously at three distant sites, with 5.106 pfu of poxvirus (MVA strain expressing the E6/E7 protein of HPV16) at D8, 15 and 22. 0.5 UI of tubertest, 5 10⁶ cfu of BCG or 0.5% of levamisole was injected subcutaneously just before each immunization over the sites of injection to the shaved skin of mice (approx. 10 cm²). Tumor growth was monitored, twice a week during 80 days, with a calliper. Mice were euthanised for ethical reasons when their tumor size was superior to 25 mm of diameter or when they showed pain even if the tumor was smaller.

For the immunogenicity study, 3 C57BI6 female mice were vaccinated subcutaneously at three distant sites with 5.10⁷ pfu of poxvirus (MVA strain) at D1, 8 and 15. This dose was used to optimize the detection of cellular immunity against HPV specific antigens. 0.5 UI of tubertest, 5 10⁶ cfu of BCG or 0.5% of levamisole was injected subcutaneously just before each immunization over the sites of injection to the shaved skin of mice (approx. 10 cm2). Spleen and serum were removed at D22 for immunological analysis.

1. Measure of the Number/Frequency of IFNγ Secreting Cells by Elispot

Fresh spleen cells were prepared using Lympholite purification buffer. All the peptides were synthesized by Neosystem at the immunograde level (10 mg). Each peptide was dissolved in DMSO at 10 mg/ml and store at 4° C. A 96-well nitrocellulose plate was coated with 3 μg/ml monoclonal rat anti-mouse IFNγ antibody (Clone R4-6A2; Pharmingen, cat. nr551216, Lot M072862; 100 μl/well) in Sodium Carbonate Buffer. The plates were incubated overnight at 4° C. or 1 h at 37° C. Plates were washed three times with DMEM 10% FCS and saturated 2 hours at 37° C. with 100 μl DMEM 10% FCS/well. Splenocytes were plated at a concentration of 106 cells/100 μl. Interleukine 2 was added to all the wells at a concentration of 6 U/50 μl/well (R&D Systems) 10 ng/ml). ConcanavalinA was used as positive control (5 μg/ml). HPV specific peptides were used at a concentration of 5 μg/ml. The plates were incubated 48 hours at 37° C., 5% CO₂. The plate was washed one time with PBS 1× and 5 times with PBS-Tween 0.05%. Biotinylated Anti-mouse IFNγ (clone XMG1.2, Pharmingen) was added at the concentration of 0.3 μg/100 μl/well and incubated 2 hours at room temperature under slow agitation. The plate was washed 5 times with PBS-Tween 0.05%. Extravidin AKP (Sigma, St. Louis, Mo.) diluted at 1/5000 in PBS-Tween0.05%-FCS1% was also added to the wells (100 μl/well). The plate was incubated 45 minutes at room temperature and then washed 5 times with PBS-Tween 0.05%. IFNγ secretion was revealed with Biorad Kit. 100 μl substrate (NBT+BCIP) was added per well and plate was left at room temperature for ½ hour. The plate was washed with water and put to dry overnight at room temperature. Spots were counted using a dissecting microscope.

2. List of Tested Peptides:

SCVYCKKEL (E6; Db): S9L Peptide

RCIICQRPL (E6; Db): R9L Peptide

SEYRHYQYS (E6; Kb): S9S Peptide

ECVYCKQQL (E6; Db): E9L Peptide

TDLHCYEQL (E7; Kb): T9L Peptide

RAHYNIVTF (E7; Db): R9F Peptide

Irrelevant Peptide (Flu specific)

3. Measure of the Frequency of R9F Tetramer Specific CD8+ T Cells

Fresh spleen cells were harvested and prepared using a BD specific sieve (Cell Strainer). Splenocytes were stimulated during 5 days with R9F peptide (5 μg/ml) in 24 well plates or used directly for specific labelling. 1.10⁶ cells were stained with 1 μl of an APC-coupled mouse CD8 specific antibody (BD Pharmingen 553035; clone 53-6.7; lot no 32567) and 10 μl of R9F specific H-2 Db tetramer (Beckman Coulter T20071; H-2 Db/PE; peptide RAHYNIVTF; lot C507117; C602110) during 30 min at 4° C. Cells were washed then diluted in PBS/0.5% PFA.

4. Results:

A therapeutic experiment has been done in the TC1 subcutaneous model as described in the protocol section. We have observed that a pre-treatment by a subcutaneous administration of 0.5 UI of PPD 5% increase significantly the therapeutic efficacy of MVATG8042 by 45% to 65% of tumor free mice at the end of the experiment (see FIG. 1). The statistical difference in in vivo survival experiment between the different groups was assessed using a Log Rank application (Statistica 5.1 software, Statsoft Inc.) of the Kaplan-Meier survival curves. A P≦0.05 is considered statistically significant. Previously described adjuvants (i.e. levamisole, live BCG), known to efficiently enhance traditional vaccines are unable to increase the therapeutic efficacy of the nucleic acid vaccine MVATG8042.

An immunogenicity study was also performed in parallel to look for the induction of cellular responses against E6 and E7 HPV antigens. Mice were vaccinated as described in the protocol section.

In a first set of experiments (see FIG. 2), the number of E6 or E7-specific IFNγ secreting cells was enumerated using an ELISPOT assay. E6 and E7H-2 Db restricted peptides were used to monitored CD8 T cell response after immunization. We have observed that pre-treatment with a subcutaneous administration of PPD improve significantly the number of MHC class I restricted CD8 T cells obtained as well as the number of H-2 Db restricted peptides recognized.

In the same way, the number of CD8+/R9F Tetramer+ T cells has also been measured by flow cytometry analysis (FIG. 3) before or after in vitro stimulation with the E7-specific immunodominant epitope R9F. Thus indicate that the recognition of the R9F immunodominant epitope is clearly mediated by CD8 specific T cells. This population is relatively low in the spleen and a flow cytometry analysis required an in vitro stimulation with the peptide. Pre-treatment with PPD improve the number of specific CD8 T cells against this particular epitope wherein pre-treatment with levamisole and live BCG are unable to do so (the observed differences are not statistically significant).

1-29. (canceled)

30. A vaccine composition comprising (i) PPD and (ii) a nucleic acid sequence encoding an antigen.

31. The vaccine composition of claim 30, wherein said PPD is a composition selected from the group consisting of tubertest and tubersol.

32. The vaccine composition of claim 30, wherein said antigen is a tumor associated antigen (TAA).

33. The vaccine composition of claim 30, wherein said antigen is a microbial antigen.

34. The vaccine composition of claim 34, wherein said microbial antigen is an antigen of a virus, a bacteria, a parasite or a fungus.

35. The vaccine composition of claim 30, wherein said antigen is an antigen of an infectious organism.

36. The vaccine composition of claim 34, wherein said antigen is selected from the group consisting of an E6 early coding region of HPV, an E7 early coding region of HPV and part or combination thereof.

37. The vaccine composition of claim 30, wherein said antigen is an allergen.

38. The vaccine composition of claim 30, wherein said nucleic acid sequence encoding an antigen is comprised in a vector.

39. The vaccine composition of claim 38, wherein said vector is of plasmid or viral origin.

40. The vaccine composition of claim 40, wherein said vector is obtained from a poxvirus, an adenovirus, a retrovirus, a herpes virus, an alpha virus, a foamy virus, or an adenovirus-associated virus.

41. The vaccine composition of claim 40, wherein said vector is obtained from a MVA.

42. The vaccine composition of claim 38, wherein said vector further comprises elements sufficient for expression of the antigen.

43. The vaccine composition of claim 30, further comprising a pharmaceutically acceptable carrier.

44. A process for the manufacture of the vaccine composition of claim 30, comprising mixing PPD with a nucleic acid sequence encoding an antigen.

45. The process of claim 44, further comprising the step of incorporating the vaccine composition within a pharmaceutically acceptable carrier.

46. A method of treating a patient suffering from or susceptible to a tumor, an infectious disease, or an allergy, comprising administering to said patient an affective amount of the vaccine composition of claim 30.

47. A method of increasing an immune response of a mammal to an antigen, said method comprising administering to the mammal (i) PPD, and (ii) a nucleic acid sequence encoding an antigen.

48. The method of claim 47, wherein said PPD and said nucleic acid sequence encoding an antigen are administered simultaneously.

49. The method of claim 47, wherein said PPD and said nucleic acid sequence encoding an antigen are administered sequentially.

50. The method of claim 47, wherein said method further comprises raising an immune response in said mammal against a disease state.

51. The method of claim 46, wherein said patient is a cancer patient undergoing chemotherapeutic treatment with a chemotherapeutic agent.

52. The method of claim 46, wherein said patient is undergoing treatment with cytotoxic drugs and/or radiotherapy, and wherein said administering of said vaccine composition improves the cytotoxic effectiveness of said cytotoxic drugs and/or radiotherapy.


SEQUENCE LISTING (6 sequences)

SEQ ID NO: 1 - protein, 9 aa, Human papillomavirus
  PEPTIDE (1)..(9)
SCVYCKKEL

SEQ ID NO: 2 - protein, 9 aa, Human papillomavirus
  PEPTIDE (1)..(9)
RCIICQRPL

SEQ ID NO: 3 - protein, 9 aa, Human papillomavirus
  PEPTIDE (1)..(9)
SEYRHYQYS

SEQ ID NO: 4 - protein, 9 aa, Human papillomavirus
  PEPTIDE (1)..(9)
ECVYCKQQL

SEQ ID NO: 5 - protein, 9 aa, Human papillomavirus
  PEPTIDE (1)..(9)
TDLHCYEQL

SEQ ID NO: 6 - protein, 9 aa, Human papillomavirus
  PEPTIDE (1)..(9)
RAHYNIVTF